(19) United States
(12) Patent Application Publication
(10) Pub. No.: US 2024/0050448 A1
(43) Pub. Date: Feb. 15, 2024

(54) Biodegradable Implant Including Naltrexone and Cholesterol

(71) Applicant: BioCorRx, Inc., Anaheim, CA (US)

(72) Inventors: Lourdes Felix, Anaheim, CA (US); Brady James Granier, Anaheim, CA (US)

(21) Appl. No.: 18/270,682

(22) PCT Filed: Dec. 30, 2021
(86) PCT No.: PCT/US21/65666

§ 371 Date: Jun. 30, 2023

Related U.S. Application Data
(60) Provisional application No. 63/132,425, filed on Dec. 30, 2020.
(60) Provisional application No. 63/134,089, filed on Jan. 5, 2021.

(51) Int. Cl. A61K 31/58; A61K 31/485; A61K 9/00; A61K 47/28; A61K 47/12

(52) U.S. Cl. CPC A61K 31/58; A61K 31/485; A61K 9/0024; A61K 47/28; A61K 47/12

(57) ABSTRACT

The disclosure provides a subcutaneous biodegradable medical implant comprising naltrexone and a cholesterol amount comprising less than about 10% cholesterol, and methods of treating a disease or disorder in a patient comprising placing the subcutaneous biodegradable medical implant in a patient.

### CROSS REFERENCE TO RELATED APPLICATIONS

This application claims the benefit of U.S. Provisional Application Ser. No. 63/132,425, titled “BIODEGRADABLE IMPLANT INCLUDING NALTREXONE AND CHOLESTEROL,” filed Dec. 30, 2020, and of U.S. Provisional Application Ser. No. 63/134,089, titled “BIODEGRADABLE IMPLANT INCLUDING NALTREXONE AND CHOLESTEROL,” filed Jan. 5, 2021, each of which is incorporated by reference herein in its entirety for all purposes.

### BACKGROUND

Naltrexone is a prescription drug belonging to a class of drugs called opioid antagonists. Applicant has identified a number of deficiencies associated with conventional administering of naltrexone, various solutions to which are described with respect to several embodiments described herein.

### BRIEF SUMMARY

In one aspect, the disclosure provides a subcutaneous biodegradable medical implant comprising naltrexone and less than about 10% cholesterol by weight, wherein the subcutaneous biodegradable medical implant is capable of releasing a dosage amount of the naltrexone from the subcutaneous biodegradable medical implant following subcutaneous placement of the subcutaneous biodegradable medical implant in a patient. The subcutaneous biodegradable medical implant is useful in preventing and treating diseases and disorders in a patient, including addictive disorders (e.g., including opioid addiction, alcohol addiction, addictive personality disorders, gaming or gambling addictions, social media addiction, screen addiction, food addition, and the like), obesity, and weight gain.

In one aspect, the invention provides a subcutaneous biodegradable medical implant comprising naltrexone and a cholesterol amount comprising less than about 10% cholesterol by weight, wherein the subcutaneous biodegradable medical implant is capable of releasing a dosage amount of the naltrexone from the subcutaneous biodegradable medical implant following placement of the subcutaneous biodegradable medical implant in a patient.

In some subcutaneous biodegradable medical implants, the cholesterol amount comprises about 0.5% cholesterol by weight, about 1% cholesterol by weight, about 1.5% cholesterol by weight, about 2% cholesterol by weight, about 2.5% cholesterol by weight, about 3% cholesterol by weight, about 3.5% cholesterol by weight, about 4% cholesterol by weight, about 4.5% cholesterol by weight, about 5% cholesterol by weight, about 5.5% cholesterol by weight, about 6% cholesterol by weight, about 6.5% cholesterol by weight, about 7% cholesterol by weight, about 7.5% cholesterol by weight, about 8% cholesterol by weight, about 8.5% cholesterol by weight, about 9% cholesterol by weight, or about 9.5% cholesterol by weight.

Some subcutaneous biodegradable medical implants are configured to release a dosage amount of naltrexone in an amount in a range of 150 mg to 5 grams into a bloodstream of the patient.

In some subcutaneous biodegradable medical implants, the cholesterol amount comprises about 2% cholesterol by weight. In some subcutaneous biodegradable medical implants, an amount of the naltrexone comprises 200 mg naltrexone base, and wherein the subcutaneous biodegradable medical implant further comprises 2 mg magnesium stearate and 4 mg cholesterol, and is free from triamcinolone or triamcinolone acetonide. In some subcutaneous biodegradable medical implants, an amount of the naltrexone comprises 200 mg naltrexone base, and wherein the subcutaneous biodegradable medical implant further comprises 2 mg triamcinolone acetonide, 2 mg magnesium stearate, and 4 mg cholesterol. In some subcutaneous biodegradable medical implants, an amount of the naltrexone comprises 1000 mg naltrexone base, and wherein the subcutaneous biodegradable medical implant further comprises 10 mg triamcinolone acetonide, 10 mg magnesium stearate, and 20 mg cholesterol.

In some subcutaneous biodegradable medical implants, the cholesterol amount comprises about 4% cholesterol by weight. In some subcutaneous biodegradable medical implants, an amount of the naltrexone comprises 200 mg naltrexone base, and wherein the subcutaneous biodegradable medical implant further comprises 2 mg magnesium stearate and 8 mg cholesterol, and is free from triamcinolone or triamcinolone acetonide. In some subcutaneous biodegradable medical implants, an amount of the naltrexone comprises 200 mg naltrexone base, and wherein the subcutaneous biodegradable medical implant further comprises 2 mg triamcinolone acetonide, 2 mg magnesium stearate, and 8 mg cholesterol. In some subcutaneous biodegradable medical implants, an amount of the naltrexone comprises 1000 mg naltrexone base, and wherein the subcutaneous biodegradable medical implant further comprises 10 mg triamcinolone acetonide, 10 mg magnesium stearate, and 40 mg cholesterol.

In some subcutaneous biodegradable medical implants, the cholesterol amount comprises about 8% cholesterol by weight. In some subcutaneous biodegradable medical implants, an amount of the naltrexone comprises 200 mg naltrexone base, and wherein the subcutaneous biodegradable medical implant further comprises 2 mg magnesium stearate and 16 mg cholesterol, and is free from triamcinolone or triamcinolone acetonide. In some subcutaneous biodegradable medical implants, an amount of the naltrexone comprises 200 mg naltrexone base, and wherein the subcutaneous biodegradable medical implant further comprises 2 mg triamcinolone acetonide, 2 mg magnesium stearate, and 16 mg cholesterol. In some subcutaneous biodegradable medical implants, an amount of the naltrexone comprises 1000 mg naltrexone base, and wherein the subcutaneous biodegradable medical implant further comprises 10 mg triamcinolone acetonide, 10 mg magnesium stearate, and 80 mg cholesterol.

In another aspect, the invention provides a method of preventing or treating a disease or disorder in a patient, comprising: placing a subcutaneous biodegradable medical implant comprising naltrexone and a cholesterol amount comprising less than about 10% cholesterol by weight in the patient, wherein the subcutaneous biodegradable medical implant is capable of releasing a dosage amount of the naltrexone from the subcutaneous biodegradable medical implant following placement of the subcutaneous biodegradable medical implant in the patient, thereby treating the disease or disorder.

In some methods, the disease or disorder is one or more of an addiction disorder, opioid addition, alcohol abuse, alcohol addiction, gambling addiction, gaming addiction, sex addiction, screen addiction, social media addiction, food addiction, obsessive-compulsive disorder, obesity, or weight gain. In some methods, the disease or disorder is associated with hypothyroidism, Hashimoto's thyroiditis, polycystic ovary syndrome (PCOS), or sleep apnea. In some methods, the disease or disorder is associated with chronic pain, inflammation, or complex regional pain syndrome.

In some methods, the cholesterol amount comprises about 0.5% cholesterol by weight, about 1% cholesterol by weight, about 1.5% cholesterol by weight, about 2% cholesterol by weight, about 2.5% cholesterol by weight, about 3% cholesterol by weight, about 3.5% cholesterol by weight, about 4% cholesterol by weight, about 4.5% cholesterol by weight, about 5% cholesterol by weight, about 5.5% cholesterol by weight, about 6% cholesterol by weight, about 6.5% cholesterol by weight, about 7% cholesterol by weight, about 7.5% cholesterol by weight, about 8% cholesterol by weight, about 8.5% cholesterol by weight, about 9% cholesterol by weight, or about 9.5% cholesterol by weight.

In some methods, the subcutaneous biodegradable medical implant comprising naltrexone and the cholesterol amount comprising less than about 10% cholesterol by weight releases a dosage amount of naltrexone in an amount in a range of 150 mg to 5 grams into a bloodstream of the patient.

In some methods, the cholesterol amount comprises about 2% cholesterol by weight. In some methods, an amount of the naltrexone comprises 200 mg naltrexone base, and wherein the subcutaneous biodegradable medical implant further comprises 2 mg magnesium stearate and 4 mg cholesterol, and is free from triamcinolone or triamcinolone acetonide. In some methods, an amount of the naltrexone comprises 200 mg naltrexone base, and wherein the subcutaneous biodegradable medical implant further comprises 2 mg triamcinolone acetonide, 2 mg magnesium stearate, and 4 mg cholesterol. In some methods, an amount of the naltrexone comprises 1000 mg naltrexone base, and wherein the subcutaneous biodegradable medical implant further comprises 10 mg triamcinolone acetonide, 10 mg magnesium stearate, and 20 mg cholesterol.

In some methods, the cholesterol amount comprises about 4% cholesterol by weight. In some methods, an amount of the naltrexone comprises 200 mg naltrexone base, and wherein the subcutaneous biodegradable medical implant further comprises 2 mg magnesium stearate and 8 mg cholesterol, and is free from triamcinolone or triamcinolone acetonide. In some methods, an amount of the naltrexone comprises 200 mg naltrexone base, and wherein the subcutaneous biodegradable medical implant further comprises 2 mg triamcinolone acetonide, 2 mg magnesium stearate, and 8 mg cholesterol. In some methods, an amount of the naltrexone comprises 1000 mg naltrexone base, and wherein the subcutaneous biodegradable medical implant further comprises 10 mg triamcinolone acetonide, 10 mg magnesium stearate, and 40 mg cholesterol.

In some methods, the cholesterol amount comprises about 8% cholesterol. In some methods, an amount of the naltrexone comprises 200 mg naltrexone base, and wherein the subcutaneous biodegradable medical implant further comprises 2 mg magnesium stearate and 16 mg cholesterol, and is free from triamcinolone or triamcinolone acetonide. In some methods, an amount of the naltrexone comprises 200 mg naltrexone base, and wherein the subcutaneous biodegradable medical implant further comprises 2 mg triamcinolone acetonide, 2 mg magnesium stearate, and 16 mg cholesterol. In some methods, an amount of the naltrexone comprises 1000 mg naltrexone base, and wherein the subcutaneous biodegradable medical implant further comprises 10 mg triamcinolone acetonide, 10 mg magnesium stearate, and 80 mg cholesterol.

The details of one or more embodiments of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

Having thus described the disclosure in general terms, reference will now be made to the accompanying drawings, which are not necessarily drawn to scale, and wherein:

FIG. 1 is a diagram of an exemplary subcutaneous implant placed in a patient according to embodiments of the present disclosure.

### DETAILED DESCRIPTION OF VARIOUS EMBODIMENTS

Various embodiments of the present disclosure now will be described more fully hereinafter with reference to the accompanying drawings, in which some, but not all embodiments of the disclosure are shown. Indeed, the disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout.

The term “biodegradable,” as used herein, refers, in one embodiment, to a material that is degraded in a biological environment. In another embodiment, “biodegradable” refers to a material that has a finite half-life in a biological environment. In another embodiment, “biodegradable” refers to a material that has a measurable half-life in a biological environment. In another embodiment, “biodegradable” refers to a material that is degraded inside a living organism. In another embodiment, “biodegradable” refers to a material that has a finite half-life inside a living organism. In another embodiment, “biodegradable” refers to a material that has a measurable half-life inside a living organism. In another embodiment, “biodegradable” refers to a material that diminishes in mass over time within a living organism.

In one embodiment, the half-life is 1 month or less. In another embodiment, the half-life is 2 months or less. In another embodiment, the half-life is 3 months or less. In another embodiment, the half-life is 4 months or less. In another embodiment, the half-life is 5 months or less. In another embodiment, the half-life is 6 months or less. In another embodiment, the half-life is 8 months or less. In another embodiment, the half-life is 10 months or less. In another embodiment, the half-life is one year or less. In another embodiment, the half-life is 1.5 years or less. In another embodiment, the half-life is 2 years or less. In another embodiment, the half-life is 3 years or less. In another embodiment, the half-life is 4 years or less. In another embodiment, the half-life is 5 years or less. In another embodiment, the half-life is 7 years or less. In another embodiment, the half-life is 10 years or less. Each possibility represents a separate embodiment of the present disclosure.

Compositions or methods “comprising” or “including” one or more recited elements may include other elements not specifically recited.

Designation of a range of values includes all integers within or defining the range, and all subranges defined by integers within the range.

Unless otherwise apparent from the context, the term “about” encompasses insubstantial variations, such as values within a standard margin of error of measurement (e.g., SEM) of a stated value. Unless otherwise apparent from the context, the term “about” encompasses values within ±5% or ±10% of a stated value.

The singular forms of the articles “a,” “an,” and “the” include plural references unless the context clearly dictates otherwise.

### A. Overview

Various embodiments of the disclosure generally relate to a subcutaneous biodegradable medical implant comprising naltrexone (e.g., naltrexone hydrochloride, naltrexone base) and less than about 10% cholesterol by weight, that, when implanted in a patient, aids in treatment of diseases and disorders in the patient. In embodiments, the subcutaneous biodegradable medical implant is a biodegradable implanted pellet. Implants of the disclosure are useful in treating an addiction disorder, including but not limited to opioid addition, alcohol use, gambling addiction, gaming addiction, sex addiction, screen addiction, social media addiction, food addiction, or obsessive-compulsive disorder. Implants of the disclosure are useful in treating obesity, weight gain, and weight gain associated with hypothyroidism, Hashimoto's thyroiditis, polycystic ovary syndrome (PCOS), or sleep apnea. Implants of the disclosure are useful in treating chronic pain, inflammation, and complex regional pain syndrome in the patient. Implants of the disclosure are useful in aiding weight loss in a patient.

The molecular formula for naltrexone is C₂₀H₂₃NO₄. It will be appreciated that naltrexone may also be referred to as Vivitrex, ReVia, N-Cyclopropylmethylnoroxymorphone, Vivitrol, Celupan, Naltrexonum, Naltrexona, Naltrel, N-Cyclopropylmethyl-14-hydroxidihydromorphinone, among others. The present application applies to the use of the identified molecular formula, regardless of what terminology is used to reference it.

It will be appreciated that the biodegradable embodiments of the present disclosure eliminate a need for physical or intentional removal of the implants from a patient. The implant comprising naltrexone may biodegrade into the bloodstream, eliminating the requirement for removal of the implant, over a varying number of days or months depending on the metabolism of the patient. The implant comprising naltrexone provides a sustained release of naltrexone into the bloodstream of the patient. The implant comprising naltrexone provides a gradually descending sustained level of release of naltrexone into the bloodstream of the patient over the course of treatment. Such sustained release of naltrexone into the bloodstream overcomes several drawbacks associated with oral-based medication administration systems.

The present implants comprising naltrexone eliminate the need for oral administration, which eliminates the need for a patient's liver to process the drug. Such a bypass is significantly beneficial for those patients with fatty liver disease and other conditions that would prohibit a patient from processing naltrexone in a healthy manner. An implant opens the door for patients who may not otherwise be candidates for treatments involving administration of naltrexone. Additionally, oral administration tends to require a higher dosage than is required when using an implant.

Further, in an oral-based naltrexone administration system, non-compliance with the medication plan is a common issue. Reasons for non-compliance include a patient forgetting to take the medication at the scheduled time (e.g., forgetting to take the medication every day; forgetting to take the medication at the same time each day) and a patient opting not to take the medication as a result of distaste for or discomfort related to possible side effects, for example gastrointestinal complaints such as diarrhea and abdominal cramping, liver damage, and more. Examples of side effects associated with oral administration of naltrexone may include symptoms of anxiety, allergic dermatitis, arthralgias, myalgias, insomnia, fatigue, skin rash, headache disorder, nausea, vomiting, abdominal pain with cramps, angioedema, among others. Such non-compliance significantly reduces likelihood of long-term success of a treatment regimen.

Patients receiving administration of naltrexone sometimes preferably meet certain physical requirements in order for the implant to be safe and successful. Examples of such physical requirements vary according to the intended treatment or indication, and may include without limitation specific liver enzyme levels, certain BMI levels, and the patient should not be taking any opioids.

Cholesterol in an implant slows the release of the naltrexone active ingredient (e.g., the cholesterol reduces absorption by creating a temporary barrier between the naltrexone active ingredient and adipose tissue and by also adding bulk mass to the overall implantable pellet in a desirable amount). An amount of cholesterol is chosen to balance the rate of release of the naltrexone from the pellet into the bloodstream of the patient over the lifetime of the pellet and to minimize the amount of remaining cholesterol in the pellet after all or most of the naltrexone is released. That is, for a given indication, the amount of cholesterol in the implant or pellet is chosen such that there is not an excess of cholesterol remaining toward the end of a lifetime of the pellet such that a less than ideal or preferred amount of naltrexone is released into or absorbed into the bloodstream of the patient toward the end of a lifetime of the pellet (e.g., too much cholesterol may inhibit the sustained release of naltrexone). At the same time, for a given indication, an amount of cholesterol in the implant or pellet is chosen such that enough cholesterol remains surrounding the pellet of naltrexone to allow for a desired release or absorption of naltrexone over the lifetime of the pellet (e.g., too little cholesterol may result in too high of a release of naltrexone at any given point in time during the lifetime of the pellet, and especially toward the end of a lifetime of the pellet). Pellets having 10% or more of cholesterol may lead to an increase in remnant residue in a patient as well as an undesirable increase in total pellet mass (e.g., this may inhibit a more desirable smaller pellet without an added benefit). Moreover, pellets having too high of an amount of cholesterol may be undesirably impacted with respect to compressibility or binding (e.g., a loss of compressibility or satisfactory binding of materials); in such examples, the cholesterol may dissolve into the blood stream and an undesirably high rate making the intended treatment ineffective.

### B. Therapeutic Uses of Naltrexone Implants

Various embodiments of the present disclosure generally relate to a subcutaneous biodegradable medical implant comprising naltrexone (e.g., naltrexone hydrochloride, naltrexone base) and less than about 10% cholesterol by weight, that, when implanted in a patient aids in treatment of a disease or disorder in the patient. Implants of the disclosure are useful in treating an addiction disorder, including but not limited to opioid addition, alcohol use, gambling addiction, gaming addiction, sex addiction, screen addiction, social media addiction, or obsessive-compulsive disorder. Implants of the disclosure are useful in treating obesity, weight gain, and weight gain associated with hypothyroidism, Hashimoto's thyroiditis. polycystic ovary syndrome (PCOS), or sleep apnea. Implants of the disclosure are useful in treating chronic pain, inflammation, and complex regional pain syndrome.

### C. Composition of Naltrexone Pellets

In some embodiments, the subcutaneous biodegradable medical implant may comprise other excipients and/or non-active ingredients as part of the manufacturing process as well as a small amount of steroid to prevent inflammation. Exemplary excipients and/or non-active ingredients may include cholesterol and magnesium stearate. Exemplary steroids for use in preventing inflammation may include triamcinolone or triamcinolone acetonide (TCA). Some pellets comprise TCA, for example about 1% TCA by weight. Some pellets according to embodiments herein are free from triamcinolone or triamcinolone acetonide; that is, some pellets according to embodiments herein do not contain any triamcinolone or triamcinolone acetonide. Some pellets comprise magnesium stearate, for example about 1% magnesium stearate by weight.

Example pellets of the present disclosure may comprise less than about 10% cholesterol by weight. Some implants comprise less than about 9%, less than about 8%, less than about 7%, less than about 6%, less than about 5%, less than about 4%, less than about 3%, less than about 2%, or less than about 1% cholesterol. Some implants comprise about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, or about 9.5% cholesterol. Some embodiments comprise about 2% cholesterol. Some embodiments comprise about 1% magnesium stearate and about 2% cholesterol. Some embodiments comprise about 1% magnesium stearate, about 1% triamcinolone acetonide, and about 2% cholesterol. Some embodiments comprise about 4% cholesterol. Some embodiments comprise about 1% magnesium stearate and about 4% cholesterol. Some embodiments comprise about 1% magnesium stearate, about 1% triamcinolone acetonide, and about 4% cholesterol. Some embodiments comprise about 8% cholesterol. Some embodiments comprise about 1% magnesium stearate and about 8% cholesterol. Some embodiments comprise about 1% magnesium stearate, about 1% triamcinolone acetonide, and about 8% cholesterol. Some implants/pellets comprise a cholesterol coating. Some implants/pellets comprise a partial coating of cholesterol on the exterior of the implant/pellet to slow an initial burst of the naltrexone active ingredient. Some embodiments comprise about 1% magnesium stearate and about 2% cholesterol. Some embodiments comprise 200 mg naltrexone base, 2 mg magnesium stearate, and 4 mg cholesterol and are free from triamcinolone or triamcinolone acetonide. Some embodiments comprise about 1% magnesium stearate, about 1% triamcinolone acetonide (steroid active ingredient), and about 2% cholesterol. Some embodiments comprise 200 mg naltrexone base, 2 mg triamcinolone acetonide (steroid active ingredient), 2 mg magnesium stearate, and 4 mg cholesterol. Some embodiments comprise 1000 mg naltrexone base, 10 mg triamcinolone acetonide (steroid active ingredient), 10 mg magnesium stearate, and 20 mg cholesterol. Some embodiments comprise about 1% magnesium stearate and about 4% cholesterol. Some embodiments comprise 200 mg naltrexone base, 2 mg magnesium stearate, and 8 mg cholesterol and are free from triamcinolone or triamcinolone acetonide. Some embodiments comprise about 1% magnesium stearate, about 1% triamcinolone acetonide (steroid active ingredient), and about 4% cholesterol. Some embodiments comprise 200 mg naltrexone base, 2 mg triamcinolone acetonide (steroid active ingredient), 2 mg magnesium stearate, and 8 mg cholesterol. Some embodiments comprise 1000 mg naltrexone base, 10 mg triamcinolone acetonide (steroid active ingredient), 10 mg magnesium stearate, and 40 mg cholesterol. Some embodiments comprise about 1% magnesium stearate and about 8% cholesterol. Some embodiments comprise 200 mg naltrexone base, 2 mg magnesium stearate, and 16 mg cholesterol and are free from triamcinolone or triamcinolone acetonide. Some embodiments comprise about 1% magnesium stearate, about 1% triamcinolone acetonide (steroid active ingredient), and about 8% cholesterol. Some embodiments comprise 200 mg naltrexone base, 2 mg triamcinolone acetonide (steroid active ingredient), 2 mg magnesium stearate, and 16 mg cholesterol. Some embodiments comprise 1000 mg naltrexone base, 10 mg triamcinolone acetonide (steroid active ingredient), 10 mg magnesium stearate, and 80 mg cholesterol.

In some embodiments, the implant releases dosage amount(s) of naltrexone into a bloodstream of the patient. In embodiments, the dosage amount(s) of naltrexone can be in an amount within the range of 150 mg to 5 g. In some embodiments, the dosage amount(s) of naltrexone is in an amount of 200 mg, 400 mg, 600 mg, 800 mg, 1 g, 1.1 g, 1.2 g, 1.3 g, 1.4 g, or 1.5 g. In some embodiments, the dosage amount(s) of naltrexone is in an amount of 2.2 g. The amount of naltrexone in a pellet varies from 150 mg to 5 g naltrexone per pellet and more usually from 200 mg −1.4 g per pellet. Some pellets comprise 200 mg, 400 mg, 600 mg, 800 mg, 1 g, 1.1 g, or 1.4 naltrexone. In some embodiments, the dosage amount(s) of naltrexone can be in an amount within the range of 250 mg to 4 g, 300 mg to 4 g, 350 mg to 4 g, 400 mg to 4 g, 450 mg to 4 g, 500 mg to 4 g, 550 mg to 4 g, 600 mg to 4 g, 650 mg to 4 g, 700 mg to 4 g, 750 mg to 4 g, 800 mg to 4 g, 850 mg to 4 g, 900 mg to 4 g, 950 mg to 4 g, 1 g to 4 g, 1.1 g to 4 g, 1.5 g to 4 g, 2 g to 4 g, 2.2 g to 4 g, 2.2 g to 3 g, 2 g to 3 g, 1.1 g to 3 g, 1 g to 3 g, 950 mg to 3 g, 900 mg to 3 g, 850 mg to 3 g, 800 mg to 3 g, 750 mg to 3 g, 700 mg to 3 g, 650 mg to 3 g, 600 mg to 3 g, 550 mg to 3 g, 500 mg to 3 g, 450 mg to 3 g, 400 mg to 3 g, 350 mg to 3 g, 300 mg to 3 g, 250 mg to 3 g, 200 mg to 3 g, 200 mg to 2 g, 250 mg to 2 g, 300 mg to 2 g, 350 mg to 2 g, 400 mg to 2 g, 450 mg to 2 g, 500 mg to 2 g, 550 mg to 2 g, 600 mg to 2 g, 650 mg to 2 g, 700 mg to 2 g, 750 mg to 2 g, 800 mg to 2 g, 850 mg to 2 g, 900 mg to 2 g, 950 mg to 2 g, 1 g to 2 g, 1.1 g to 2 g, 1.5 g to 2 g.

Non-limiting examples of dosage amount(s) of naltrexone in the presently disclosed implant include any dosage or amount in increments and/or combinations of 50 mg, 100 mg, 150 mg, 200 mg, 400 mg, 500 mg, 600 mg, 700 mg, 800 mg, 900 mg, 1 g, 1.1 g, 1.4 g, and the like. It will be appreciated that dosages or amounts incrementally between those described above are within the scope of the present disclosure.

The subcutaneous medical implant may comprise a single implant unit (or otherwise referred to as a pellet) configured to release a dosage amount of the naltrexone into a bloodstream of the patient. For example, for a subcutaneous biodegradable medical implant configured to release a dosage amount of 400 mg of naltrexone into a patient's bloodstream, a single 400 mg biodegradable naltrexone pellet may be used.

The subcutaneous medical implant may comprise a plurality of implant units configured to release a dosage amount of the naltrexone into a bloodstream of the patient. In some embodiments, the subcutaneous biodegradable medical implant comprises two or more implant units (or otherwise referred to as pellets). For example, for a subcutaneous biodegradable medical implant configured to release a dosage amount of 400 mg of naltrexone into a patient's bloodstream, two (2) 200 mg biodegradable naltrexone pellets may be used.

### D. Shape, Number, and Insertion of Naltrexone Implants

In some embodiments, the subcutaneous biodegradable medical implants comprise one or more pellets formed of naltrexone and cholesterol in amounts described herein. In some embodiments, the present implants are tablet shaped, capsule shaped, rod-shaped, spherical, or cylindrical in shape. In some embodiments, the implants are approximately spherical in shape, wherein the diameter and height are approximately the same. In some embodiments, implants are cylindrical in shape, about 10 mm in diameter and about 10-11 mm in height.

A rate of release of naltrexone from the implant(s) into the patient's bloodstream is also varied by shape and number of pellets in a patient's treatment regimen. For example, an approximately spherical shaped pellet, with a smaller surface area than a rod-shaped pellet of the same volume, would release naltrexone more slowly than a rod-shaped pellet of the same volume. For example, implanting a single larger pellet would result in slower naltrexone release than multiple smaller pellets comprising the same total naltrexone dose as the single larger pellet.

Some treatment regimens utilize pellet shapes and sizes which are compatible with smaller patient incisions (e.g., incisions through which the pellets are subcutaneously placed within the patient body). Some patient incisions are closed with stitches or with Steri-Strips. In some patients, smaller incisions and closure of incisions with Steri-Strips result in reduced pain for the patient from the procedure.

The subcutaneous biodegradable medical implant may be placed, injected, or inserted below a skin surface of the patient or may be placed or injected above a muscle fascia of the patient.

In embodiments of the present disclosure, an implant comprising naltrexone is placed, injected, or inserted just beneath a surface of the skin in a lower abdominal area or hip area or other area of a patient. In some embodiments, the subcutaneous biodegradable medical implant is placed below a skin surface of a lower abdomen of the patient. In some embodiments, the subcutaneous biodegradable medical implant is placed below a skin surface of one or more of a hip, a leg, a back, and an arm of the patient. In some embodiments the implants disclosed herein are placed below a skin surface of a patient and above a muscle fascia of the patient. It will be appreciated that a placement location within a patient for a subcutaneous biodegradable medical implant is not limited to the examples herein and may vary according to a given indication or treatment plan.

E. Release into Bloodstream and Biodegradation of Pellets, Dosage Frequency

In embodiments, the subcutaneous biodegradable medical implant biodegrades in the patient. In some embodiments, the subcutaneous biodegradable medical implant biodegrades after a period of about 30 days in the patient. In embodiments, the subcutaneous biodegradable medical implant biodegrades over a period of about several months in the patient.

In an example, naltrexone is released from the implant into the bloodstream of a patient over a period of about 4 weeks to one year. In an example, naltrexone is released from the implant into the bloodstream of a patient over a period of about 4 weeks, 5 weeks, 6 weeks, 8 weeks, 9 weeks, 10 weeks, 11 weeks, 12 weeks, one month, 2 months, 3 months, 4 months, 5 months, 6 months, 7 months, 8 months, 9 months, 10 months, 11 months, 12 months, or one year. In an example, the implant biodegrades after a period of about 30 days, 4 weeks, 5 weeks, 6 weeks, 8 weeks, 9 weeks, 10 weeks, 11 weeks, 12 weeks, one month, 2 months, 3 months, 4 months, 5 months, 6 months, 7 months, 8 months, 9 months, 10 months, 11 months, 12 months, or one year in the patient. It will be appreciated that the time it takes to for an implant to biodegrade in a patient is dependent upon multiple factors including dosage, patient metabolism, external activity, and the like. In some embodiments, a second subcutaneous biodegradable medical implant is placed into a patient subsequent to a biodegradation time of a first subcutaneous biodegradable medical implant.

Exemplary timing of implants being inserted into a patient includes once every 4 weeks, once every 5 weeks, once every 6 weeks, once every 7 weeks, once every 8 weeks, once every 9 weeks, once every 10 weeks, once every 11 weeks, once every 12 weeks, once a month, once every 2 months, once every 3 months, once every 4 months, once every 5 months, once every 6 months, once every 7 months, once every 8 months, once every 9 months, once every 10 months, once every 11 months, once every 12 months, once a year, or according to any other schedule determined by empirical analysis. Alternatively, insertions of naltrexone implants can be on an irregular basis as indicated by monitoring of symptoms of an addiction disorder, or by monitoring weight gain. It will be appreciated that, according to any given indication, a single insertion of a naltrexone implant described herein may be preferred (e.g., that is, replacement of the implant is not necessary to remain within the scope of the present disclosure).

### F. Example of a Naltrexone Pellet of the Disclosure

In an example, a subcutaneous biodegradable medical implant (pellet) comprises 200 mg naltrexone base, 2 mg magnesium stearate, and 4 mg cholesterol, with a total mass of 206 mg and is free from triamcinolone or triamcinolone acetonide. In an example, a subcutaneous biodegradable medical implant (pellet) comprises 200 mg naltrexone base, 2 mg magnesium stearate, and 8 mg cholesterol, with a total mass of 210 mg, and is free from triamcinolone or triamcinolone acetonide. In an example, a subcutaneous biodegradable medical implant (pellet) comprises 200 mg naltrexone base, 2 mg magnesium stearate, and 16 mg cholesterol, with a total mass of 218 mg, and is free from triamcinolone or triamcinolone acetonide.

In an example, a subcutaneous biodegradable medical implant (pellet) comprises 200 mg naltrexone base, 2 mg triamcinolone acetonide (e.g., a steroid active ingredient), 2 mg magnesium stearate, and 4 mg cholesterol, with a total mass of 208 mg. In an example, a subcutaneous biodegradable medical implant (pellet) comprises 200 mg naltrexone base, 2 mg triamcinolone acetonide (steroid active ingredient), 2 mg magnesium stearate, and 8 mg cholesterol, with a total mass of 212 mg. In an example, a subcutaneous biodegradable medical implant (pellet) comprises 200 mg naltrexone base, 2 mg triamcinolone acetonide (steroid active ingredient), 2 mg magnesium stearate, and 16 mg cholesterol, with a total mass of 220 mg.

Pellets comprising 400 mg, 1.1 g and 1.4 g of naltrexone may be produced using the same or similar ratios of those exemplary components described above with respect to the 200 mg pellets (e.g., 1% magnesium stearate, 1% triamcinolone acetonide (steroid active ingredient), and 2% cholesterol; 1% magnesium stearate and 2% cholesterol; 1% magnesium stearate, 1% triamcinolone acetonide (steroid active ingredient) and 4% cholesterol; 1% magnesium stearate and 4% cholesterol; 1% magnesium stearate, 1% triamcinolone acetonide (steroid active ingredient) and 8% cholesterol; 1% magnesium stearate and 8% cholesterol).

In an example, a subcutaneous biodegradable medical implant (pellet) comprises 1000 mg naltrexone base, 10 mg triamcinolone acetonide (steroid active ingredient), 10 mg magnesium stearate, and 20 mg cholesterol, with a total mass of 1040 mg. In an example, a subcutaneous biodegradable medical implant (pellet) comprises 1000 mg naltrexone base, 10 mg triamcinolone acetonide (steroid active ingredient), 10 mg magnesium stearate, and 40 mg cholesterol, with a total mass of 1060 mg. In an example, a subcutaneous biodegradable medical implant (pellet) comprises 1000 mg naltrexone base, 10 mg triamcinolone acetonide (steroid active ingredient), 10 mg magnesium stearate, and 80 mg cholesterol, with a total mass of 1100 mg.

### G. Exemplary Placement of Naltrexone Implant in a Patient

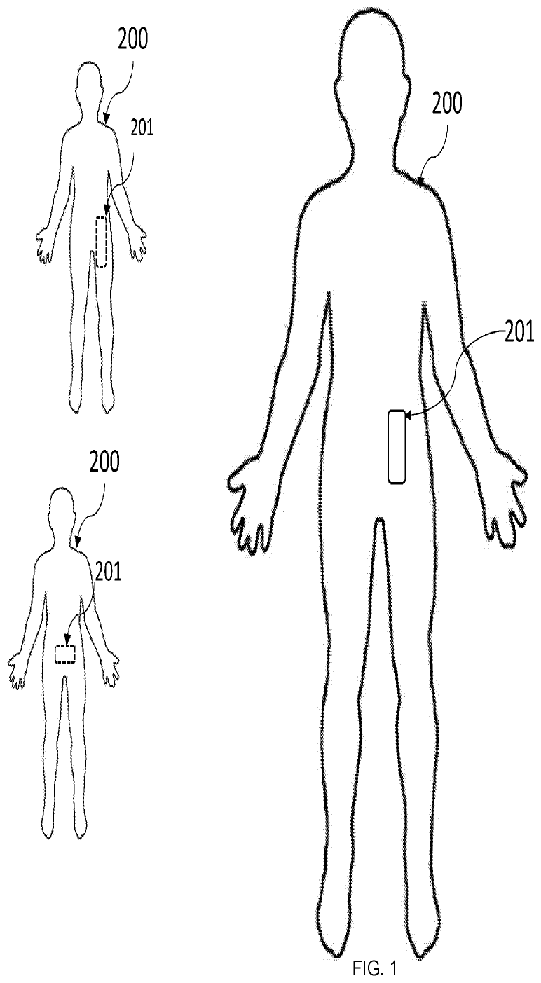

In an example, the subcutaneous biodegradable medical implant is placed in a patient according to FIG. 1.

FIG. 1 is a diagram of an exemplary subcutaneous implant placed in a patient according to embodiments of the present disclosure. In embodiments of the present disclosure, a subcutaneous biodegradable medical implant comprising naltrexone 201 is placed into a patient 200. It will be appreciated that, while implant 201 is shown as having been placed into an abdominal area of patient 200, embodiments including placement of the implant into other areas of patient 200 are within the spirit of the present disclosure (e.g., lower abdominal area, hip area, as shown in FIG. 1). It will also be appreciated that implant 200 is not drawn to scale in FIG. 1.

A subcutaneous biodegradable medical implant may be inserted using an insertion device (e.g., a syringe, an applicator, a trocar, or any other appropriate insertion device).

### H. Exemplary 72-Hour Dissolution Study of a Naltrexone Pellet of the Disclosure

Dissolution of 1000 mg naltrexone base pellets comprising varying amounts of cholesterol (0 mg, 20 mg, 40 mg, and 80 mg cholesterol) was measured after 8, 24, 48, and 72 hours in phosphate-buffered saline (PBS)+20% ethanol pH 5.5, 900 ml 37° C. buffer. The dissolution was performed in triplicate using one pellet per chamber and the studies were run over 8, 24, 48, and 72 hours using a PBS +20% ethanol pH 5.5, 900 ml 37° C. buffer. Released naltrexone was measured by high performance liquid chromatography (HPLC). The pH of the medium was measured after the ethanol is added so it was an apparent pH.

Composition of naltrexone pellets was as shown in Table 1.

TABLE 1
Naltrexone Pellets used in 72-hour Dissolution Study

| | | % Cholesterol |
|---|---|---|
| Batch No. | Composition | by weight |
| TE21L2901 | Naltrexone base 1000 mg | 0% |
| | Triamcinolone Acetonide 10 mg | |
| | Magnesium Stearate 10 mg | |
| TE21L2902 | Naltrexone base 1000 mg | 2% |
| | Triamcinolone Acetonide 10 mg | |
| | Magnesium Stearate 10 mg | |
| | Cholesterol 20 mg | |
| TE21L2903 | Naltrexone base 1000 mg | 4% |
| | Triamcinolone Acetonide 10 mg | |
| | Magnesium Stearate 10 mg | |
| | Cholesterol 40 mg | |
| TE21L2904 | Naltrexone base 1000 mg | 8% |
| | Triamcinolone Acetonide 10 mg | |
| | Magnesium Stearate 10 mg | |
| | Cholesterol 80 mg | |

Results of 72-hour dissolution study are shown in Table 2.

TABLE 2
Results of 72-hour Dissolution Study (Cylindrical pellets
about 10 mm in diameter and about 10-11 mm in height.)

| Serial | | | |
|---|---|---|---|
| No | Batch No | Time(Hours) | % Release |
| 1. | TE21L2901 | 8 | 4.42 |
| | TE21L2901 | 8 | 4.51 |
| | TE21L2901 | 8 | 5.75 |
| 2. | TE21L2902 | 8 | 5.93 |
| | TE21L2902 | 8 | 6.08 |
| | TE21L2902 | 8 | 6.32 |
| 3. | TE21L2903 | 8 | 5.19 |
| | TE21L2903 | 8 | 6.28 |
| | TE21L2903 | 8 | 6.31 |
| 4. | TE21L2904 | 8 | 6.68 |
| | TE21L2904 | 8 | 6.55 |
| | TE21L2904 | 8 | 6.29 |
| 5. | TE21L2901 | 24 | 14.64 |
| | TE21L2901 | 24 | 15.25 |
| | TE21L2901 | 24 | 15.15 |
| 6. | TE21L2902 | 24 | 13.72 |
| | TE21L2902 | 24 | 15.09 |
| | TE21L2902 | 24 | 14.30 |
| 7. | TE21L2903 | 24 | 11.32 |
| | TE21L2903 | 24 | 12.58 |
| | TE21L2903 | 24 | 12.14 |
| 8. | TE21L2904 | 24 | 17.77 |
| | TE21L2904 | 24 | 16.52 |
| | TE21L2904 | 24 | 17.67 |
| 9 | TE21L2901 | 48 | 18.90 |
| | TE21L2901 | 48 | 22.31 |
| | TE21L2901 | 48 | 21.88 |
| 10. | TE21L2902 | 48 | 14.80 |
| | TE21L2902 | 48 | 15.15 |
| | TE21L2902 | 48 | 15.27 |
| 11. | TE21L2903 | 48 | 13.77 |
| | TE21L2903 | 48 | 16.82 |
| | TE21L2903 | 48 | 17.43 |
| 12. | TE21L2904 | 48 | 17.88 |
| | TE21L2904 | 48 | 17.83 |
| | TE21L2904 | 48 | 17.94 |
| 13. | TE21L2901 | 72 | 27.61 |
| | TE21L2901 | 72 | 29.62 |
| | TE21L2901 | 72 | 29.95 |
| 14. | TE21L2902 | 72 | 22.78 |
| | TE21L2902 | 72 | 22.35 |
| | TE21L2902 | 72 | 22.77 |
| 15. | TE21L2903 | 72 | 20.72 |
| | TE21L2903 | 72 | 20.59 |
| | TE21L2903 | 72 | 21.27 |
| 16. | TE21L2904 | 72 | 23.14 |
| | TE21L2904 | 72 | 23.15 |
| | TE21L2904 | 72 | 24.67 |

Conclusion from 72-Hour Dissolution Study:

Pellets containing 2%, 4%, or 8% cholesterol showed slowed release of naltrexone, compared to 0% cholesterol pellets, in 72-hour dissolution study. The 4% cholesterol formulation performed with the most desirable release profile. However 2% and 8% formulations were still in a desirable range. The slower release profile of the 4% cholesterol formulation compared to the 8% cholesterol formulation was surprising as it had been expected that the more cholesterol in the pellet, the slower the pellet would dissolve until compressibility was affected, however dissolution of the 8% cholesterol pellet was faster than that of the 4% cholesterol pellet.

### CONCLUSION

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

1. A subcutaneous biodegradable medical implant comprising naltrexone and a cholesterol amount comprising less than about 10% cholesterol by weight, wherein the subcutaneous biodegradable medical implant is capable of releasing a dosage amount of the naltrexone from the subcutaneous biodegradable medical implant following placement of the subcutaneous biodegradable medical implant in a patient.

2. The subcutaneous biodegradable medical implant of claim 1, wherein the cholesterol amount comprises about 0.5% cholesterol by weight, about 1% cholesterol by weight, about 1.5% cholesterol by weight, about 2% cholesterol by weight, about 2.5% cholesterol by weight, about 3% cholesterol by weight, about 3.5% cholesterol by weight, about 4% cholesterol by weight, about 4.5% cholesterol by weight, about 5% cholesterol by weight, about 5.5% cholesterol by weight, about 6% cholesterol by weight, about 6.5% cholesterol by weight, about 7% cholesterol by weight, about 7.5% cholesterol by weight, about 8% cholesterol by weight, about 8.5% cholesterol by weight, about 9% cholesterol by weight, or about 9.5% cholesterol by weight.

3. The subcutaneous biodegradable medical implant of claim 1, configured to release a dosage amount of naltrexone in an amount in a range of 150 mg to 5 grams into a bloodstream of the patient.

4. The subcutaneous biodegradable medical implant of claim 2, wherein the cholesterol amount comprises about 2% cholesterol by weight.

5. The subcutaneous biodegradable medical implant of claim 1, wherein an amount of the naltrexone comprises 200 mg naltrexone base, and wherein the subcutaneous biodegradable medical implant further comprises 2 mg magnesium stearate and 4 mg cholesterol, and is free from triamcinolone or triamcinolone acetonide.

6. The subcutaneous biodegradable medical implant of claim 1, wherein an amount of the naltrexone comprises 200 mg naltrexone base, and wherein the subcutaneous biodegradable medical implant further comprises 2 mg triamcinolone acetonide, 2 mg magnesium stearate, and 4 mg cholesterol.

7. The subcutaneous biodegradable medical implant of claim 1, wherein an amount of the naltrexone comprises 1000 mg naltrexone base, and wherein the subcutaneous biodegradable medical implant further comprises 10 mg triamcinolone acetonide, 10 mg magnesium stearate, and 20 mg cholesterol.

8. The subcutaneous biodegradable medical implant of claim 2, wherein the cholesterol amount comprises about 4% cholesterol by weight.

9. The subcutaneous biodegradable medical implant of claim 1, wherein an amount of the naltrexone comprises 200 mg naltrexone base, and wherein the subcutaneous biodegradable medical implant further comprises 2 mg magnesium stearate and 8 mg cholesterol, and is free from triamcinolone or triamcinolone acetonide.

10. The subcutaneous biodegradable medical implant of claim 1, wherein an amount of the naltrexone comprises 200 mg naltrexone base, and wherein the subcutaneous biodegradable medical implant further comprises 2 mg triamcinolone acetonide, 2 mg magnesium stearate, and 8 mg cholesterol.

11. The subcutaneous biodegradable medical implant of claim 1, wherein an amount of the naltrexone comprises 1000 mg naltrexone base, and wherein the subcutaneous biodegradable medical implant further comprises 10 mg triamcinolone acetonide, 10 mg magnesium stearate, and 40 mg cholesterol.

12. The subcutaneous biodegradable medical implant of claim 2, wherein the cholesterol amount comprises about 8% cholesterol by weight.

13. The subcutaneous biodegradable medical implant of claim 1, wherein an amount of the naltrexone comprises 200 mg naltrexone base, and wherein the subcutaneous biodegradable medical implant further comprises 2 mg magnesium stearate and 16 mg cholesterol, and is free from triamcinolone or triamcinolone acetonide.

14. The subcutaneous biodegradable medical implant of claim 1, wherein an amount of the naltrexone comprises 200 mg naltrexone base, and wherein the subcutaneous biodegradable medical implant further comprises 2 mg triamcinolone acetonide, 2 mg magnesium stearate, and 16 mg cholesterol.

15. The subcutaneous biodegradable medical implant of claim 1, wherein an amount of the naltrexone comprises 1000 mg naltrexone base, and wherein the subcutaneous biodegradable medical implant further comprises 10 mg triamcinolone acetonide, 10 mg magnesium stearate, and 80 mg cholesterol.

16. A method of preventing or treating a disease or disorder in a patient, comprising:
placing a subcutaneous biodegradable medical implant comprising naltrexone and a cholesterol amount comprising less than about 10% cholesterol by weight in the patient, wherein the subcutaneous biodegradable medical implant is capable of releasing a dosage amount of the naltrexone from the subcutaneous biodegradable medical implant following placement of the subcutaneous biodegradable medical implant in the patient, thereby treating the disease or disorder.

17. The method of claim 16, wherein the disease or disorder is one or more of an addiction disorder, opioid addition, alcohol abuse, alcohol addiction, gambling addiction, gaming addiction, sex addiction, screen addiction, social media addiction, food addiction, obsessive-compulsive disorder, obesity, or weight gain.

18. The method of claim 16, wherein the disease or disorder is associated with hypothyroidism, Hashimoto's thyroiditis, polycystic ovary syndrome (PCOS), or sleep apnea.

19. The method of claim 16, wherein the disease or disorder is associated with chronic pain, inflammation, or complex regional pain syndrome.

20. The method of any of claims 16-19, wherein the cholesterol amount comprises about 0.5% cholesterol by weight, about 1% cholesterol by weight, about 1.5% cholesterol by weight, about 2% cholesterol by weight, about 2.5% cholesterol by weight, about 3% cholesterol by weight, about 3.5% cholesterol by weight, about 4% cholesterol by weight, about 4.5% cholesterol by weight, about 5% cholesterol by weight, about 5.5% cholesterol by weight, about 6% cholesterol by weight, about 6.5% cholesterol by weight, about 7% cholesterol by weight, about 7.5% cholesterol by weight, about 8% cholesterol by weight, about 8.5% cholesterol by weight, about 9% cholesterol by weight, or about 9.5% cholesterol by weight.

21. The method of any of claims 16-20, wherein the subcutaneous biodegradable medical implant comprising naltrexone and the cholesterol amount comprising less than about 10% cholesterol by weight releases a dosage amount of naltrexone in an amount in a range of 150 mg to 5 grams into a bloodstream of the patient.

22. The method of claim 20, wherein the cholesterol amount comprises about 2% cholesterol by weight.

23. The method of any of claims 16-20, wherein an amount of the naltrexone comprises 200 mg naltrexone base, and wherein the subcutaneous biodegradable medical implant further comprises 2 mg magnesium stearate and 4 mg cholesterol, and is free from triamcinolone or triamcinolone acetonide.

24. The method of any of claims 16-20, wherein an amount of the naltrexone comprises 200 mg naltrexone base, and wherein the subcutaneous biodegradable medical implant further comprises 2 mg triamcinolone acetonide, 2 mg magnesium stearate, and 4 mg cholesterol.

25. The method of any of claims 16-20, wherein an amount of the naltrexone comprises 1000 mg naltrexone base, and wherein the subcutaneous biodegradable medical implant further comprises 10 mg triamcinolone acetonide, 10 mg magnesium stearate, and 20 mg cholesterol.

26. The method of claim 20, wherein the cholesterol amount comprises about 4% cholesterol by weight.

27. The method of any of claims 16-20, wherein an amount of the naltrexone comprises 200 mg naltrexone base, and wherein the subcutaneous biodegradable medical implant further comprises 2 mg magnesium stearate and 8 mg cholesterol, and is free from triamcinolone or triamcinolone acetonide.

28. The method of any of claims 16-20, wherein an amount of the naltrexone comprises 200 mg naltrexone base, and wherein the subcutaneous biodegradable medical implant further comprises 2 mg triamcinolone acetonide, 2 mg magnesium stearate, and 8 mg cholesterol.

29. The method of any of claims 16-20, wherein an amount of the naltrexone comprises 1000 mg naltrexone base, and wherein the subcutaneous biodegradable medical implant further comprises 10 mg triamcinolone acetonide, 10 mg magnesium stearate, and 40 mg cholesterol.

30. The method of claim 20, wherein the cholesterol amount comprises about 8% cholesterol.

31. The method of any of claims 16-20, wherein an amount of the naltrexone comprises 200 mg naltrexone base, and wherein the subcutaneous biodegradable medical implant further comprises 2 mg magnesium stearate and 16 mg cholesterol, and is free from triamcinolone or triamcinolone acetonide.

32. The method of any of claims 16-20, wherein an amount of the naltrexone comprises 200 mg naltrexone base, and wherein the subcutaneous biodegradable medical implant further comprises 2 mg triamcinolone acetonide, 2 mg magnesium stearate, and 16 mg cholesterol.

33. The method of any of claims 16-20, wherein an amount of the naltrexone comprises 1000 mg naltrexone base, and wherein the subcutaneous biodegradable medical implant further comprises 10 mg triamcinolone acetonide, 10 mg magnesium stearate, and 80 mg cholesterol.
